(19) United States
(12) Patent Application Publication
(10) Pub. No.: US 2014/0276980 A1
(43) Pub. Date: Sep. 18, 2014

(54) WOUND CLOSURE DEVICE AND METHOD

(71) Applicant: PATAMRA, LLC, Glendale, AZ (US)

(72) Inventors: Patrick Smith, Glendale, AZ (US); Tomas James North, Marana, AZ (US)

(21) Appl. No.: 13/841,986

(22) Filed: Mar. 15, 2013

(51) Int. Cl. A61B 17/04

(52) U.S. Cl. CPC A61B 17/0469
USPC 606/144

(57) ABSTRACT

A wound closure device and method are disclosed. The wound closure device includes a housing, a needle driver assembly, a needle guard assembly, and optionally, a bolster. Use of the device and method reduces the length of time it takes to successfully close a wound or incision when compared to manual suturing as well as decreases the risk of complications such as internal organ damage and hemorrhage caused by the suture needles and eventual herniation through the abdominal wall caused by a poorly and ineffectively closed wound or incision.

### FIELD OF THE INVENTION

The present invention relates, generally, to wound closure devices and methods, and more particularly, the invention relates to suturing devices and methods for closing wounds or incisions.

### BACKGROUND OF THE INVENTION

Laparoscopic surgery is a minimally invasive approach to exploring, diagnosing, and treating disease within the body and requires a limited number of small incisions to gain access to a body cavity. The benefits of laparoscopic surgery, when compared to traditional open procedures that require much larger incisions resulting in blood loss and transfusions, include lower risk of infection, reduction in post-operative pain and use of narcotic analgesia, shortened recovery time, less post-operative scarring, and the like. Although laparoscopic procedure times are usually slightly longer when compared to traditional open surgery, hospital stay has been shown to be shorter due to reduced recovery time and need for intravenous narcotic analgesia. Additional concerns regarding laparoscopic surgery include slightly longer operation times and associated anesthesia exposure, risk of organ and major blood vessel perforation due to use of a trocar, and post-operative herniation through the port sites due to ineffective wound closure and avoidance of wound closure due to technical difficulty. Another consideration is that, as a result of the technical difficulty associated with laparoscopic surgery, many surgeons have not routinely attempted to close smaller laparoscopic port wounds at all, particularly those less than 10 mm. In this case patients are at an even higher risk of, for example, abdominal wall herniation, which can result in repeat procedures and exposure to a further cycle of associated complications.

Traditional examples where laparoscopic surgery has been beneficial include cholecystectomy (over 1 million performed in the United States annually), colectomy, appendectomy, hysterectomy, ovarian cyst removal, tubal ligation, and thoracic surgery known as VATS (Video Assisted Thorascopic Surgery) procedures. In addition recent developments in robotics have led to automated laparoscopic tools, such as the ‘Da Vinci’ system whereby the surgeon is entirely removed from the patient and the laparoscopic tools are driven remotely via an advanced control system. The market and sophistication of laparoscopic surgeries has advanced considerably in recent years with the total number performed in the United States estimated to exceed 4 million procedures annually. However, port wound closure is still generally carried out in the same manner as it was over 3 decades ago when the first laparoscopic appendectomy was performed.

Some of the specific challenges a surgeon faces during laparoscopic surgery include: 1) port wounds result in fascial defects that are approximately 5-20 mm in diameter; 2) the tough fascial tissue at the area to be sutured is located at the bottom of a long tissue tunnel, making it very difficult to visualize and grasp in order to suture and close the wound; 3) port wound closure with current methods using a curved or straight needle and suture are imprecise, time consuming, technically and physically demanding and often result in abdominal wall hernias leading to repeat surgeries which are compounded by the attendant increase in costs to both the patient and the healthcare system and likely in the near future the hospital when considering the movement towards captivated care being driven by Medicare and third-party payers.

In the past decade there have been a number of attempts to address the issue of port wound closure with rapidly deployable, simple to use suturing devices. Such devices include those described in United States Application No. 20100145364 to Keren et al., issued Jun. 10, 2010, entitled “Suture Device;” U.S. Pat. No. 7,771,438 to Dreyfuss et al., issued Aug. 10, 2010, entitled “Suture Passer;” U.S. Pat. No. 6,743,241 to Kerr, issued Jun. 1, 2004, entitled “Laparoscopic Port Site Fascial Closure Device;” and United States Application No. 20090292300 to Hamilton et al., issued Nov. 26, 2009, entitled “Suturing Device, System, and Method.” Each of these devices exhibit strengths and weakness; however, three common problems appear consistently among them all. First, the highly technical and complicated design of each raises the question of reliability in terms of potential manufacturing difficulties and utilization defects during the procedure. Second, the cost of manufacture, maintenance, and disposal of each device carries a cost burden that may prove to be less than economical when attempting to deploy in the marketplace. Third, each of these devices when deployed to close a port wound expose organs and large blood vessels to open ended needles, thereby increasing the risk for perforation, hemorrhage, and in the case of abdominal procedures, bowel obstruction.

As stated previously the technical difficulty with port wound closure and subsequent herniation carries a number of indirect yet costly implications to both the patient and the hospital. The period of time it takes a surgeon to close a series of laparoscopic port wounds may be as long as 30-45 minutes depending on the body habitus of the patient. The longer time it takes to close the wound may imply that it's more technically difficult and thus more likely to end in an ineffective closure and subsequent herniation even in the months or years to follow. Concerning the extended length of time it takes to close laparoscopic port wounds, the average opportunity cost in the operating room is typically $600 per minute. This represents an area of potentially significant cost savings by reducing time in the operating room.

### SUMMARY OF THE INVENTION

The present invention generally relates to a device and method for closing or suturing surgical incisions or other wounds. While the ways in which the present invention addresses the drawbacks of the prior art are discussed in greater detail below, in general, the device and method 1) significantly reduce the length of time is takes to close surgical port and other wounds; 2) address potential complications arising from the use of similar devices, such as perforation, hemorrhage, and in the case of abdominal procedures bowel obstruction; and 3) decrease the likelihood of post-operative complications such as herniation. In addition, the device and method, in accordance with various exemplary embodiments of the invention, provide design efficiencies directed at minimizing a number of parts incorporated into the device, thereby reducing a likelihood of manufacturing attributable defects; these same design efficiencies translate into reduced production, maintenance, and disposal costs.

In accordance with various embodiments of the invention, a wound closure device includes a housing, a needle driver assembly, a needle guard assembly, and optionally, a bolster. In accordance with various aspects of these embodiments, the needle driver assembly includes one or more needles (e.g., two needles; e.g., single bodied or multi-bodied, segmented configuration), one or more sutures coupled to the needles, such that each needle is coupled to a suture, and a needle driver for each needle. In accordance with further aspects, the needle guard assembly includes one or more needle guards, wherein each needle guard may include an insert, a needle guard actuator, and a link to cause the needle guards protract from the housing and toward an insertion end of the device.

In accordance with additional embodiments of the invention, a wound closure device includes a housing comprising a first end and a second end distal the first end and at least one lumen therein, a needle driver assembly comprising at least one needle driver at least partially within the at least one lumen, and a retractable needle guard assembly, the retractable needle guard assembly comprising one or more needle guards proximate the second end, each needle guard comprising a link end coupled to a link and a free end distal the link end, and a needle guard actuator to cause the one or more needle guards to protract by causing the free end to extend from the housing and away from the first end. In accordance with various aspects of these embodiments, the lumen includes a curvature to guide the needle driver. In accordance with additional aspects, the device further comprises an insert in the needle guard to engage with the needle. The insert may be formed of flexible material or mesh and may be replaceable. In accordance with further aspects, the housing may be formed of multiple parts, some of which may be reused to close additional wounds.

In accordance with additional embodiments of the invention, a method of using a wound closure device includes the steps of providing a wound closure device having a housing, a needle driver assembly, a needle guard assembly, including at least one needle guard, and optionally, a bolster; activating a needle guard assembly to protract the needles guards; and activating a needle driver assembly to drive one or more needles from an interior portion of the housing to engage with a needle guard or an insert. In accordance with additional aspects of these embodiments, a method further includes the step of retracting a needle driver to close and retract the needle guards. In accordance with various aspects of these embodiments, an ‘above fascial’ approach is employed, which uses symmetric needle guards below to form a ‘closed loop’ suturing method, prohibiting the exposure of internal organs and blood vessels to sharp needles, thereby greatly reducing the risk of perforation, hemorrhage, and in the case of abdominal procedures bowel obstruction.

In accordance with yet further embodiments of the invention, a method of using a wound closure device includes the steps of providing a wound closure device comprising a housing having a first end and a second end distal the first end, a needle driver assembly, and a needle guard assembly; activating the needle guard assembly to cause one or more needle guards proximate the second end to protract away from the housing and toward the second end; and activating the needle driver assembly to cause a first needle to move toward the needle guard and become engaged with the needle guard or insert. In accordance with various aspects of these embodiments, the method further comprises a step of retracting the needle guard to cause the needle guard to rotate toward the housing and the first end.

In accordance with yet additional embodiments of the invention, a kit includes at least one device, including a housing, a needle driver assembly, a needle guard assembly, and optionally, a bolster. In accordance with various aspects of these embodiments, a kit includes a plurality of disposable devices. In accordance with additional aspects, a kit includes a first portion of the device and a plurality of disposable second portions, which each include a needle guard assembly and needles. In accordance with yet further aspects, the kit includes one or more devices and a plurality of inserts, needles, and sutures.

In accordance with additional embodiments of the invention, a kit includes a device including a housing comprising a first end and a second end distal the first end and at least one lumen therein; a needle driver assembly comprising at least one needle driver at least partially within the at least one lumen; and a retractable needle guard assembly, the retractable needle guard assembly comprising one or more needle guards proximate the second end, each needle guard comprising a link end coupled to a link and a free end distal the link end, and a needle guard actuator to cause the one or more needle guards to protract by causing the free end to extend from the housing and away from the first end. In accordance with various aspects of these embodiments, the kit includes one or more cartridges, including one or more needles and sutures. In accordance with additional aspects, the kit includes a plurality of single-use devices. In accordance with further aspects, the kit includes a plurality of sutures, a plurality of needles, and a plurality of inserts.

These and other features and advantages of the present invention will become apparent upon a reading of the following detailed description when taken in conjunction with the drawing figures, wherein there is shown and described various illustrative embodiments of the invention.

### BRIEF DESCRIPTION OF THE DRAWING FIGURES

The exemplary embodiments of the present invention will be described in connection with the appended drawing figures in which like numerals denote like elements and:

FIG. 1 illustrates a wound closure device in accordance with various embodiments of the invention;

FIG. 2 illustrates a cutaway view of a wound closure device in accordance with various embodiments of the invention;

FIG. 3 illustrates a cutaway view of a top portion of a wound closure device in accordance with various embodiments of the invention;

FIG. 4 illustrates a cutaway view of a bottom portion of a wound closure device in accordance with various embodiments of the invention;

FIG. 5 illustrates a cutaway view of a bottom portion of a wound closure device, showing protraction of needle guards, in accordance with various embodiments of the invention;

FIGS. 6(a)-9(b) illustrate use of a wound closure device in accordance with additional embodiments of the invention;

FIG. 10 illustrates a wound closure device in accordance with additional exemplary embodiments of the invention;

FIG. 11 illustrates a cross-sectional view of the wound closure device illustrated in FIG. 10; and

FIGS. 12(a) and 12(b) illustrated the wound closure device illustrated in FIG. 10 with protracted needle guards.

It will be appreciated that elements in the figures are illustrated for simplicity and clarity and have not necessarily been drawn to scale. For example, the dimensions of some of the elements in the figures may be exaggerated relative to other elements to help to improve understanding of illustrated embodiments of the present invention.

### DETAILED DESCRIPTION OF THE INVENTION

The description of exemplary embodiments of the present invention provided below is merely exemplary and is intended for purposes of illustration only; the following description is not intended to limit the scope of the invention disclosed herein. Moreover, recitation of multiple embodiments having stated features is not intended to exclude other embodiments having additional features or other embodiments incorporating different combinations of the stated features.

The present invention provides an improved wound closure device and method. As set forth in more detail below, the wound closure device and method can be used to suture or otherwise close a variety of wounds and are particularly well suited for closure of laparoscopic port hole incisions to rapidly, securely, and safely close the port (e.g., a trocar) hole. However, the device and method may also be suited for other types of wound closures, such as arthroscopic or similar types of incisions or wounds. The device and method described herein can be used to reduce the length of time it takes to close a wound, compared to manual suturing, as well as decrease the risk of complications such as internal organ damage caused by the suturing needles and eventual herniation through the abdominal wall caused by a poorly and ineffectively closed wound. Further, the design of the device of the present disclosure is relatively uncomplicated, reliable, and easy to use compared to similar devices. In addition, as discussed in more detail below, use of the device is relatively safe, compared to other devices. The device and method described herein may be used on a variety of animals, and are particularly well suited for humans and other mammals.

FIGS. 1-5 illustrate a wound closure device 100 in accordance with various exemplary embodiments of the invention. Device 100 includes a housing 102; a needle guard assembly 104, including a needle guard actuator 106, one or more needle guards 108—each needle guard 108 including an portion or insert 402 to engage with a needle 404, and a link system 202 to facilitate protraction and retraction of needle guards 108 away from and toward housing 102; a needle driver assembly 204, including one or more needle drivers 302, one or more needles 404, and a needle driver actuator or grip 110; and device 100 optionally includes a bolster 112. Device 100 includes a first end 114 and a distal second or insertion end 116.

Housing 102 may be of any suitable shape, and in the embodiment illustrated in FIGS. 1-5 is generally in the shape of a hollow cylinder. Dimensions of housing 102 may vary in accordance with various uses or applications of the device; for example, the diameter may vary from sizes ranging from approximately 5-20 mm outside diameter, about 1-2 mm wall thickness, and the length of hosing may vary from about 100-250 mm or more, depending on the application. By way of examples, device 100 may be inserted through a 5-20 mm port hole that runs through the wall of the abdomen and opens to the abdominal cavity. Housing 102 and device 100 may also be scaled for pediatric procedures. Device 100 may alternatively be designed for veterinary uses. The distal or insertion end of device 100 may include a tapered or conical shape with a blunt tip to enable ease of insertion through the port hole while minimizing the risk of damage to internal organs during the insertion process.

Housing 102 may be formed of any suitable material. Device 100 and housing 102 may incorporate knurled and/or surface manufacturing techniques upon the external surface of the housing 102 to allow the surgeon to maintain a secure grip on device 100.

Housing 102 includes an interior cavity to receive needle guard actuator 106 and includes lumens 406 to receive needle drivers 302. Needles 404 and needle drivers 302 may initially lie within lumens 406 within housing 102; alternatively needles 404 and optionally a portion of needle drivers 302 may reside outside lumens 406. In the illustrated example, housing 102 includes two lumens 406, which lie in a plane and are symmetric at about 180 degrees about the center axis of device 100. By way of one example, each lumen 406 is approximately 1 mm in diameter, runs the length of housing 102, towards a distal or insertion end 116 of housing 102, and curves externally such that lumens 406 terminated at an external wall 408 of housing 102 proximal to needle guard 108, when guards 108 are in a retracted position. When lumens 406 include a curvature away from the center axis of device 100 near distal end, as illustrated in FIGS. 2, 4, and 5, needles 404 and needle drivers 302 may exit sides of the device 100 (e.g., through tissue) away from housing 102. Of note, the non-memory or assembled conformation of the exemplary (e.g., nitinol) needle drivers 302 is such that distal ends of needle drivers 302 follow the curvature of the lumens 406. Lumens 406 serve to hold and guide needle drivers 302 during device 100 actuation. Lumens 406 may have any suitable shape, such as a hollow cylinder or the like.

Needle driver assembly 204 includes needle drivers 302, needle driver actuator 110, needles 404, and sutures (not illustrated, which may include traditional, non-absorbable, or bioabsorbable sutures). In the illustrated embodiment, device 100 includes two needle drivers 302. By way of one example, needle drivers 302 are thin-walled tubes approximately 1 mm OD and 0.5 mm ID composed of a memory metal such as nitinol; however, needle drivers 302 may be formed of other materials, such as plastic and other metals. In one example, the memory position is that of a linear conformation; however driver 302 retains the flexibility to allow for bending as it moves away from wall 408 of housing 102. Once away from housing 102, needle driver 302 regains its linear conformation as it pushes needle 404 and the suture through, e.g., a fascia, directing needle 404 into an engaged position with portion or insert 402 of needle guard 108. Needle driver actuator 110 is mechanically coupled to needle drivers 302 and allows an operator (e.g., a surgeon or robotic mechanism) to apply and transmit axial force to the needle driver assembly—e.g. along a direction from first end 114 toward second end 116. Needle driver actuator 110 may incorporate a rigid ‘loop’ or flange onto the side of the external surface of needle driver actuator 110, enabling secure transmission of axial force from the surgeons thumb or finger to the needle driver assembly 110 during actuation of device 100.

Needles 404 may range from, for example, size 1-5, be straight or curved, incorporate a barb near the cutting end and attached suture at the non-cutting end, and range in length from about 2-10 mm. The barb may be configured to enable secure engagement with portion or insert 402. In accordance with one example, needle 404 sits within a bore of needle driver 302 protruding from the distal end having the attached suture run the length of the bore of needle driver 302 with excess extending beyond the proximal end. In accordance with other embodiments, the suture need not be within an interior of housing 102. Needle 404 may include a lip on a bottom portion to facilitate coupling (e.g., using a snap fit) of needle 404 to needle driver 302. This allows for rapid replacement of needles 404 and sutures (which may be preloaded onto needles 404). Further, needles 404 may include a curvature when not in lumen 406, to create a tension fit between needle 404 within lumen 406. Another embodiment utilizes a mechanism whereby needle 404 is segmented and comprised of individual linked rods attached end to end enabling a curved trajectory as the needle assembly traverses the fascia on its course to engagement with portion or insert 402 of needle guard 108. Another embodiment does not utilize needles 404 and suture(s), but instead a bio-absorbable polymer material that secures the wound.

Referring now to FIGS. 1-2 and 4-5, exemplary needle guard assembly 104 includes a plurality of needle guards 108, each including portion or insert 402, one or more links 410-418 (which form part of link system 202), and pins 504-512, which allow needle guards 108 to rotate from an angle of about 0 degrees (e.g., prior to activating needle guard actuator 106) to respective angles of about −90 degrees and +90 degrees (e.g., after needle guard actuator 106 is engaged), such that the needles guards 108 are angled about 180 degrees relative to each other upon activation of needle guard actuator 106. A direction of rotation is illustrated in FIG. 5. In the illustrated example, needle guard assembly 104 includes two needle guards 108 adjoined at their bases by common pin 506. The action of protraction and retraction of the two needle guards occurs via a combination of linear translation and rotation of an ‘H’-link 502 about its pinned ends 504-508, resulting from axial movement of needle actuator 110. ‘H’-link 502 engages needle guard actuator 106 to the needle guard assembly while operating in a combined translational and rotary motion in order to facilitate smooth protraction and retraction of the needle guards 108. In the illustrated example, needle guards 108 are linked at their base by common pin 506 that is coupled to distal, tapered tip of housing 102, thereby securing needle guard assembly 104 to housing 102.

Needle guards 108 may be configured in a variety of shapes. In accordance with the illustrated example, needle guards 108 are configured to protect the organs and vessels within, for example, the abdominal cavity. By way of particular example, each needle guard 108 is approximately 15 mm long and shaped such that at least a portion of needle guard 108 fits into a cut out space at the distal end of the housing 102 (e.g., prior to activation) and substantially maintains continuity with the shape of the housing 102. At the distal tip of each guard a cutout allows for the suture to extend beyond and externally to the device 100. Needle guards 108 may also include a concavity on the inner surface of needle guard 108 that includes a recess that enables insert 402 to lie securely within the concavity. Another embodiment entails the external and internal surfaces of needle guards 108 conforming to a straight configuration. And, yet another embodiment entails the external and internal surfaces of needle guards 108 conforming to a curved configuration.

Portion or insert 402 may be disposable components and lie within the internal base of needle guards 108. In accordance with exemplary embodiments, portions or inserts 402 function to engage and secure the barb at the cutting end of the needles 404, such that when needle guards 108 are retracted prior to withdrawal of device 100 from the abdominal cavity, the attached suture(s) may then be drawn through the fascia and out through the wound along with device 100.

Optional bolster 112 is configured to provide stability to device 100 relative to a patient. Bolster 112 generally includes a protrusion extending from housing 102. By way of particular example, bolster 112 is a discoid shaped protrusion approximately 3 mm thick, 10 mm inside diameter and 30 mm outside diameter. In accordance with various embodiments of the invention, bolster 112 engages the outside surface of the housing 102 at approximately mid-shaft.

Referring now to FIGS. 6(a)-9, in accordance with exemplary embodiments of the invention, during operation or use, device 100 is inserted into a wound site 602 or a port hole (e.g., a trocar or other device used during laparoscopic or similar surgery, not illustrated) and through, e.g., an abdominal wall 604, having a fascia layer 606. In the illustrated embodiment, two opposing needle guards 108 are initially in a tucked-away position as device 100 is inserted through wall 604. Once needle guards 108 are inserted past fascia layer 606, needle guards 108 are protracted from the distal end 116 of device 100 to, e.g., ±90 degrees, as illustrated in FIGS. 7(a) and 7(b), by applying pressure to needle guard actuator 106 in the direction of needle guards 108. As illustrated, needle guards 108 protract by causing free ends 610, 612 of guards 108 to extend from the housing and away from the first end 114. Gentle partial withdrawal of device 100 is then performed, such that the protracted needle guards 108 come to lie in contact with a surface 614 wall 604 of the abdominal cavity, as illustrated in FIGS. 8(a) and 8(b). If device 100 includes bolster 112, device may then be secured in place by sliding externally located bolster 112 along the axis of the device 100 and against a surface 902 of external wall 604 of the abdomen. In this example, device 100 may stand freely and perpendicular to the wall 604 of the abdominal cavity. Next, two needles 404, along with their respective (e.g., tubular, nitinol) needle drivers 302, are forced out of or away from device 100 and through facial layer 606 of wall 604 by applying an axial force to the needle driver actuator 110 in the direction of, for example, wound site 602, as illustrated in FIGS. 9(a) and 9(b). To close or suture wound site 602, an operator (e.g., a surgeon) applies an axial force to the needle driver actuator 110 towards wound site 602, causing needles 404 to move away from device 100 at an angle, through, e.g., facial tissue 606, and directed towards portions or inserts 402 of needle guards 108. By way of one example, needles 404 travel in a straight line towards their respective needle guards 108 due to (e.g., nitinol) needle drivers 302 returning to their original axial, memory conformation. This return to an axial conformation directs needles 404 into their respective portions or inserts 402. In accordance with various exemplary embodiments of the invention, needles 404 each have a barb at the cutting end and become securely held by portions or inserts 402. To complete the wound closure, once needles 404 are engaged in their respective needle guards 108, device 100 may be moved forward through wound site 602 towards the abdominal cavity, needle guards 108 and accompanying portions or inserts 402 with engaged needles 404 and sutures are retracted to their original axial position (as illustrated in FIGS. 6(a) and 6(b)) by retracting needle guard actuator 110 away from needle guards 108, and device 100 is withdrawn from the wound, which in turn draws the suture through fascia layer 606, wall 604, and back out through the wound. The sutures are then cut from the device 100, releasing the free ends of suture which may then be tied by the surgeon, enabling secure closure of the abdominal wall wound or incision. Inserts 402 may then be removed along with needles 404 and disposed of prior to performing another wound closure on the same patient, or, in accordance with alternative embodiments, device 100 may be disposed of and a new device 100 may be used to close additional wounds on the same patient. In accordance with various exemplary embodiments, a mechanism whereby two sutures, once securely enclosed within the housing 102 following retraction of the needle guards 108, are secured to one another by means of an automated tying mechanism. In accordance with other embodiments, a mechanism whereby multiple sutures, once securely enclosed within the housing 102 following retraction of needle guards 108, are secured to one another by means of an automated crimping mechanism utilizing bio-absorbable polymers. In accordance with yet additional embodiments, a method of using device 100 includes withdrawing device 100 from the body cavity pulling the suture in the opposite direction distally through the fascial tissue, towards and into the body cavity. And, in accordance with other embodiments, a method of using device 100 includes attaching a suture, wherein the attached suture is directed from the body cavity up and out through the wound, such that both the attached and distal free ends of each suture lie outside the wound. Another embodiment incorporates a ‘hook’ and ‘loop’ feature to enable rapid and effective securing of the free ends of the suture together.

FIGS. 10-12(b) illustrate another exemplary wound closure device 1000 in accordance with additional embodiments of the invention. Device 1000 is similar to device 100, and similar parts and components of device 1000 may be the same or similar to corresponding components of device 100, unless stated otherwise herein. Device 1000 includes a housing 1002; a needle guard assembly 1004, and a needle driver assembly 1006.

Similar to housing 102, housing 1002 include a first end 1008 and a second or insertion end 1010. Housing 1002 further includes at least one lumen 1102 for a needle driver and at least one cavity 1104 for a needle guard driver or actuator. As illustrated, lumen 1102 may include a curvature. By way of example, the curvature may be between about 130 mm and 170 mm or about 150 mm. Further, housing 1002 may be formed of multiple sections as described above.

Needle guard assembly 1004 includes an actuator 1012, one or more needle guards 1014, which includes a portion or an insert 1106 to engage with a needle 1202, and one or more links 1108-1118 of a link system 1210 to facilitate protraction and retraction of needle guards 1014 from housing 1002, as described above. Needle guard assembly 1004 is similar to needle guard assembly 104, except needle guard assembly 1004 includes an additional link and a stop 1016 to prevent needle guards 1014 from protracting beyond a desired angle—e.g., about ±90 degrees from a fully retracted position of needle guards 1014. As best illustrated in FIG. 12(b), each needle guard includes a link end 1206 coupled to one or more links 1108-1114 and a free end 1208 distal the link end, such that needle guard actuator 1006 can cause the one or more needle guards to protract by causing free end 1208 to extend from housing 1002 and away from first end 1008. If included, insert 1106 may be formed of any of the materials described above in connection with insert 402, and insert 1106 may be replaceable as described above.

Needle driver assembly 1006, includes one or more needle drivers 1204 at least partially within at least one lumen 1102, one or more needles 1202, and a needle driver actuator or grip 1018.

Operation of device 1000 may be the same as operation of device 100 described above.

Referring to FIGS. 1-12(b), in accordance with the present disclosure may be configured in a variety of ways, depending on, for example the intended use for the devices (e.g., devices 100, 1000). Below are additional exemplary configurations for devices in accordance with the present disclosure.

1) One embodiment includes a single surgery, multi-use, disposable device incorporating a one piece housing attached to a needle guard assembly that in turn incorporates a disposable insert secured within each needle guard. Upon closure of a single wound (e.g., port hole), the inserts may be disposed of and new inserts may be placed in the needle guards, enabling the closure of subsequent wounds in the same patient with the original device. Upon closing all wounds, the device may be disposed of.

2) Another embodiment entails a single surgery, multi-use, disposable device (e.g., device 100 or 1000) incorporating a two-piece housing (e.g., housing 102 or 1002) whereby a detachable distal portion of the housing is attached to a needle guard assembly that in turn incorporates a disposable insert secured within each needle guard. Upon closure of a single wound, the detachable distal portion of the housing may be disposed of and a new distal portion of the housing may be attached to the proximal housing enabling the closure of subsequent wounds with the original device. Upon closing all wounds, the device may be disposed of.

3) Another embodiment entails a kit comprising a plurality of single surgery, single-use, disposable devices (e.g., devices 100 or 1000), where upon utilizing the device to close a single wound, the entire device is disposed of, and a new device is utilized in the closure of a single subsequent wound, and so on, accordingly utilizing new single-use, disposable devices for all remaining port holes.

4) Another embodiment incorporates disposable suture and needle cartridges or bobbins, enabling rapid reloading of the device.

5) Kits may include one or more devices and one or more needles and one or more sutures. A kit may also include a plurality of inserts.

The present invention has been described above with reference to a number of exemplary embodiments and examples. It should be appreciated that the particular embodiments shown and described herein are illustrative of the invention and its best mode and are not intended to limit in any way the scope of the invention. It will be recognized that changes and modifications may be made to the exemplary embodiments without departing from the scope of the present invention. These and other changes or modifications are intended to be included within the scope of the present invention.

We claim:

1. A wound closure device comprising:
a housing comprising a first end and a second end distal the first end and at least one lumen therein;
a needle driver assembly comprising at least one needle driver at least partially within the at least one lumen; and
a retractable needle guard assembly, the retractable needle guard assembly comprising one or more needle guards proximate the second end, each needle guard comprising a link end coupled to a link and a free end distal the link end, and a needle guard actuator to cause the one or more needle guards to protract by causing the free end to extend from the housing and away from the first end.

2. The wound closure device of claim 1, wherein the lumen comprises a curvature that extends from an interior of the housing to an exterior of the housing.

3. The wound closure device of claim 1, wherein curvature has a radius of about 130 mm to about 170 mm.

4. The wound closure device of claim 1, further comprising an insert.

5. The wound closure device of claim 4, wherein the insert comprises a mesh.

6. The wound closure device of claim 4, wherein the insert comprises a flexible elastomeric material.

7. The wound closure device of claim 4, wherein the insert is replaceable.

8. The wound closure device of claim 1, further comprising a needle coupled to the at least one needle driver.

9. The wound closure device of claim 8, wherein the needle initially resides in the at least one lumen prior to activation of the device.

10. The wound closure device of claim 1, further comprising a needle driver actuator proximate the first end.

11. The wound closure device of claim 1, further comprising a needle guard actuator proximate the first end.

12. The wound closure device of claim 1, wherein the housing comprises two pieces.

13. A method of using a wound closure device, the method comprising the steps of:
providing a wound closure device comprising a housing having a first end and a second end distal the first end, a needle driver assembly, and a needle guard assembly;
activating the needle guard assembly to cause one or more needle guards proximate the second end to protract away from the housing and toward the second end; and
activating the needle driver assembly to cause a first needle to move toward the needle guard and become engaged with the needle guard.

14. The method of claim 13, further comprising the step of retracting the needle guard to cause the needle guard to rotate toward the housing and the first end.

15. The method of claim 13, wherein the step of activating the needle driver assembly comprises causing the first needle to move toward a first needle guard and a second needle to move toward a second needle guard.

16. A kit including one or more wound closure devices, each wound closure device comprising:
a housing comprising a first end and a second end distal the first end and at least one lumen therein;
a needle driver assembly comprising at least one needle driver at least partially within the at least one lumen; and
a retractable needle guard assembly, the retractable needle guard assembly comprising one or more needle guards proximate the second end, each needle guard comprising a link end coupled to a link and a free end distal the link end, and a needle guard actuator to cause the one or more needle guards to protract by causing the free end to extend from the housing and away from the first end.

17. The kit of claim 16, further comprising one or more cartridges, each cartridge comprising one or more needles and one or more sutures.

18. The kit of claim 16, further comprising a plurality of inserts, a plurality of needles, and a plurality of sutures.

19. The kit of claim 16, comprising a plurality of single use devices.

20. The kit of claim 16, further comprising a plurality of inserts, wherein the plurality of inserts comprises a material selected from the group consisting of mesh and flexible elastomeric material.
